(19) United States
(12) Patent Application Publication
(10) Pub. No.: US 2023/0364115 A1
(43) Pub. Date: Nov. 16, 2023

(54) NOVEL PSYCHEDELIC COMPOSITIONS, DELIVERY SYSTEMS AND THERAPEUTIC USES THEREOF

(71) Applicant: Mydecine Innovations Group Inc., Denver, CO (US)

(72) Inventors: John C Gustin, Carlsbad, CA (US); Michael J Mccarthy, Olathe, KS (US)

(21) Appl. No.: 18/029,543

(22) PCT Filed: Oct. 1, 2021
(86) PCT No.: PCT/US2021/053136

§ 371 Date: Mar. 30, 2023

Related U.S. Application Data
(60) Provisional application No. 63/086,455, filed on Oct. 1, 2020.
(60) Provisional application No. 63/086,464, filed on Oct. 1, 2020.
(60) Provisional application No. 63/086,432, filed on Oct. 1, 2020.
(60) Provisional application No. 63/086,477, filed on Oct. 1, 2020.

(51) Int. Cl. A61K 31/675; A61K 31/4045; A61K 31/70; A61K 31/7042; A61K 31/192; A61K 31/53; A61K 31/19; A61K 31/196; A61K 31/427; A61K 31/277; A61K 31/015; A61K 31/352; A61K 31/437; A61K 31/165; A61K 31/4409; A61K 31/137; A61K 31/15; A61K 31/135; A61K 31/5375; A61K 31/421

(52) U.S. Cl. CPC A61K 31/675; A61K 31/4045; A61K 31/70; A61K 31/7042; A61K 31/192; A61K 31/53; A61K 31/19; A61K 31/196; A61K 31/427; A61K 31/277; A61K 31/015; A61K 31/352; A61K 31/437; A61K 31/165; A61K 31/4409; A61K 31/137; A61K 31/15; A61K 31/135; A61K 31/5375; A61K 31/421

(57) ABSTRACT

The present invention provides compositions and methods for the treatment of a serotonin receptor related disease or condition in a subject in need thereof. A composition of the invention includes at least one psychedelic compound, which is preferably a serotonin receptor agonist, and at least one secondary agent that modulates the activity of the serotonin receptor agonist, or the physiological response to the serotonin receptor agonist in the subject.

### CROSS-REFERENCE TO RELATED APPLICATIONS

This International PCT application claims the benefit of and priority to U.S. Provisional Application No.'s, 63/086,432 filed Oct. 1, 2020, and 63/086,455 filed Oct. 1, 2020, and 63/086,464 filed Oct. 1, 2020, and 63/086,477 filed Oct. 1, 2020. The specifications, claims and drawings of which are all incorporated herein by reference in their entirety.

### TECHNICAL FIELD

The present invention is in the technical fields of medicine and biochemistry, and in particular systems, methods, and compositions for the novel therapeutic uses of psychedelic compounds. The present invention further include improved systems, methods, and compositions for the delivery of therapeutic psychedelic compounds.

### BACKGROUND OF THE INVENTION

The serotonin 2A receptor (5-HT2A), among others has been implicated in mental disorders with complex etiologies that are still not clearly understood, in processes such as learning and memory and also in neurogenesis. Psychedelic compounds, such as psilocybin and psilocin are known agonists of the 5-HT2A serotonin receptor. Activation of this receptor has been shown to provide benefit in therapies that address mental health disorders. Given the extensive localization of this receptor to brain areas that mediate cognitive functions and social interaction, studies suggest that the 5-HT2A receptor might be involved in diseases in which these functions are impaired (generally referred to as serotonin receptor related disease(s) or condition(s)). However, due to rapid metabolism and clearance of psilocin in the body, only a small amount of active compound is able to act on a serotonin receptor. As a result, there is a need to provide improved methods, and compositions to inhibit the metabolic breakdown and clearance of a psychedelic compound, such as psilocin, as well as improved methods, and compositions to modulate the activity of the physiological response to the psychedelic compound in the subject, such as the breakdown of serotonin released as a result of the activation of a serotonin receptor.

There also exist a need to provide improved and novel methods and devices for the efficient and controlled delivery of psychedelic compounds, such as psilocybin and psilocin in therapeutic settings. More specifically, novel delivery systems that may be configured to deliver levels of psychedelic compounds, such as psilocybin and psilocin that do not induce, or strongly induce an intoxicating effect on a subject in a therapeutic or other setting. Such delivery methods would optimally be time-release in nature and further may be administered transdermally. Additional delivery methods may include intranasal delivery.

### SUMMARY OF THE INVENTION

The present inventions provide compositions and methods for the treatment of a serotonin receptor related disease or condition in a subject in need thereof. In one preferred aspect, the composition of the invention includes at least one psychedelic compound, which is preferably includes a serotonin receptor agonist, and at least one secondary agent that modulates the activity of the serotonin receptor agonist, or the physiological response to the serotonin receptor agonist in a subject, and preferably a human subject. In this aspect, a secondary agent that modulates the activity of the serotonin receptor agonist may include a compound that inhibits one or more steps of the metabolic pathway that leads to the breakdown and clearance of the agonist from the body. In this manner, the serotonin receptor agonist remains in its active form and in the body for a longer period of time, which in turn allows for lower dosages of the serotonin receptor agonist to produce the same or better therapeutic response. This improvement also allows for a more pronounced or extended therapeutic response to the serotonin receptor agonist in the subject.

In another preferred aspect, the composition of the invention includes at least one psychedelic compound and at least one secondary agent that inhibits the metabolic breakdown and clearance of the psychedelic compound in the subject.

In another preferred aspect, the composition of the invention includes at least one psychedelic compound, preferably a serotonin receptor agonist, and at least one secondary agent that modulates the activity of the physiological response to the psychedelic compound in the subject.

In another preferred aspect, the composition of the invention includes at least one psychedelic compound, preferably a serotonin receptor agonist, and at least one secondary agent that inhibits the metabolic breakdown and clearance of the psychedelic compound in the subject, and another secondary agent that modulates the activity of the physiological response to the psychedelic compound in the subject.

In another preferred aspect, the composition of the invention includes at least one psychedelic compound, which preferably includes psilocybin and/or psilocin, and at least one secondary agent, which preferably includes a UGT inhibitor that inhibits the metabolic breakdown and clearance of psilocin in the subject, and in particular through the inhibition of the glucuronidation of psilocin in the subject.

In another preferred aspect, the composition of the invention includes at least one psychedelic compound, which preferably includes psilocybin and/or psilocin, and at least one secondary agent, which preferably includes an MAOI, that modulates the activity of the physiological response to the psychedelic compound in the subject, namely inhibiting the metabolism and breakdown of serotonin released in response to activation of the 5-HT2A by psilocin.

In another preferred aspect, the composition of the invention includes at least one psychedelic compound, which preferably includes psilocybin and/or psilocin, and at least one secondary agent, which preferably includes a UGT inhibitor that inhibits the metabolic breakdown and clearance of psilocin in the subject, namely by the inactivation of psilocin by UGT-mediated glucuronidation, and at least one secondary agent, which preferably includes a MAOI, that modulates the activity of the physiological response to the psychedelic compound in the subject, namely inhibiting the metabolism and breakdown of serotonin released in response to activation of the 5-HT2A by psilocin by inhibiting the activity of monoamine oxidase in a subject.

In another preferred aspect, one or more compositions of the invention may include pharmaceutical compositions or kits containing pharmaceutical compositions prescribing information for the composition, and a container that may be used to treat a serotonin related disease of condition in a subject in need thereof.

In another preferred aspect, the invention may include methods of treating a serotonin related disease of condition in a subject in need thereof, comprising the steps of administering a therapeutically effective amount of one or more of the compositions of the invention, which may preferably include pharmaceutical compositions of the invention, or pharmaceutically acceptable salts thereof.

In another preferred aspect, one or more compositions of the invention may include pharmaceutical compositions or kits containing pharmaceutical compositions prescribing information for the composition, and a container that may be used to treat a serotonin related disease of condition in a subject in need thereof, and preferably through a transdermal delivery of the composition. In certain preferred aspects, the transdermal administration may be delivered transdermally at a therapeutically effective amount that include a non- or sub-intoxicating dose.

In another preferred aspect, one or more compositions of the invention may include pharmaceutical compositions or kits containing pharmaceutical compositions prescribing information for the composition, and a container that may be used to treat a serotonin related disease of condition in a subject in need thereof, and preferably through a intranasal delivery of the composition. In certain preferred aspects, the transdermal administration may be delivered transdermally at a therapeutically effective amount that include a non- or sub-intoxicating dose.

Additional aspects of the invention may be evidenced from the specification, claims and figures provided below.

### BRIEF DESCRIPTION OF THE FIGURES

FIG. 1: shows an exemplary metabolism profile for the an exemplary psychedelic composition and secondary agent in one embodiment thereof.

FIG. 2: shows an exemplary co-administration scheme of a therapeutically effective amount of psilocin and a UGT inhibitor in one embodiment thereof.

FIG. 3: shows an exemplary administration scheme of an synthetic psilocin in one embodiment thereof.

FIG. 4: shows an exemplary co-administration scheme of a therapeutically effective amount of an synthetic psilocin and a UGT and MAO inhibitor in one embodiment thereof.

FIG. 5: shows an exemplary transdermal administration scheme of synthetic psilocin in one embodiment thereof.

FIG. 6: shows an exemplary transdermal co-administration scheme of a therapeutically effective amount of a synthetic psilocin and a UGT and MAO inhibitor in one embodiment thereof.

FIG. 7: shows an exemplary intranasal administration scheme of psilocin in one embodiment thereof.

FIG. 8: shows an exemplary intranasal co-administration scheme of a therapeutically effective amount of psilocin and a MAO inhibitor in one embodiment thereof.

FIG. 9: demonstrates the in vivo oxidative and metabolic pathway of psilocybin in a subject.

### DETAILED DESCRIPTION OF THE INVENTION

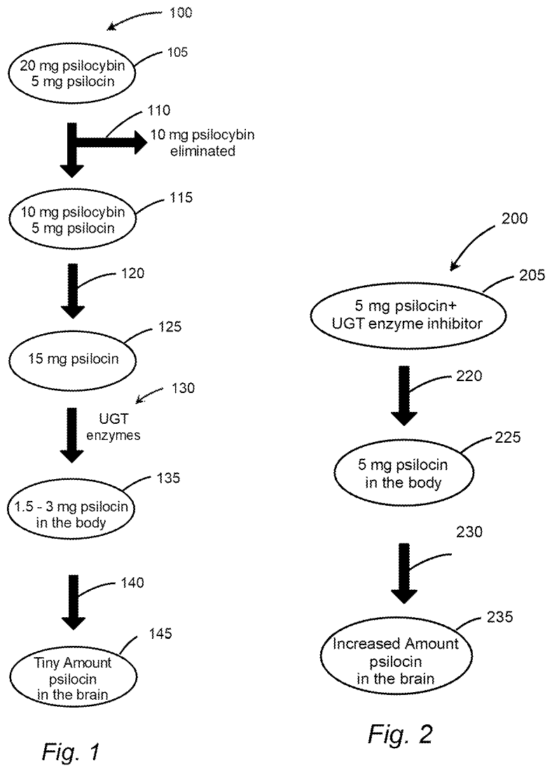

As shown generally in FIG. 1, a pathway of administration (100) of a therapeutically effective amount of an exemplary psychedelic composition having an example quantity of 20 mg psilocybin and 5 mg psilocin (105). With oral administration, about 50% of the psilocybin is eliminated from the body including by action of UGT enzymes and UGT subtypes UGT1A10 and UGT1A9 (110). The remaining psilocybin is metabolized to psilocin by alkaline phosphatase enzymes and other enzymes. Psilocin is considered the most active psychedelic compound and the more active agent between psilocybin and psilocin. Psilocin is about 10 times or greater, more active than psilocybin at inducing a therapeutic psychedelic experience. Action of UGT enzymes on psilocin results in 80% to 90% of psilocin being metabolized to inactive metabolites or removed from the body within about 30 min. The remaining psilocin is found distributed throughout the body and not know to concentrate in local regions. The result is the amount of psilocin that reaches the brain and can bind to the 5-HT2A receptor is remarkably small. Further shown in FIG. 1, is the enhancement of psilocin concentrations through protection from metabolic enzymes of the UGT enzyme family, including UGT1A9 and UGT1A10, including by inhibition of the UGT enzymes and enzyme subtypes. FIG. 1 also describes the enhancement of psilocin concentration and activity by inhibition of MAO enzymes, including MAO-A or MAO-B subtype of enzyme. In one preferred embodiment of the invention, MAO-B is inhibited prior to, or concurrently with administration of a psychedelic compounds, and in particular psilocin and/or psilocybin. Notably, MAO-B does not act directly on psilocin, but metabolizes the breakdown of dopamine which is elevated by action of psilocin activation of the 5-HT2A receptor.

As shown in the embodiment of FIG. 2, a pathway of administration (200) of a composition of the invention includes 5 mg of psilocin and a UGT enzyme inhibitor (205). In one embodiment, the UGT inhibitor may include inhibition of the UGT enzyme, preferably selective inhibition of UGT1A10 or UGT1A9 or both. As further shown in FIG. 2, administration of the therapeutically effective amount of an exemplary psychedelic composition and a UGT inhibitor allows the full 5 mg of the psilocin to become distributed in the body such that more active compounds are available to bind to and activate the 5-HT2A receptors in a subject in need thereof. With partial inhibition of UGT enzyme, the amount of psilocin need to produce a desired physiological effect is proportionally reduced. The psilocin, or effect of the psilocin is also deactivated (metabolized) by MAO enzymes, particularly by MAO-A enzymes. As such, in one preferred embodiment one or more MAO-A inhibitors are also provided in the therapeutically effective amount treatment for further enhancement of psilocin activities (230). In certain embodiments, the amount or dose of psilocin needed to achieve a therapeutic effect or a psychedelic experience is reduced, in some 5 times or more, through application of UGT inhibitor(s), MAO-A inhibitor(s), or both.

Non-limiting examples of MAO inhibitors also referred to herein as MAOIs, include MAO-A-selective inhibitors, MAO-B- selective inhibitors, and MAO non-selective inhibitors. Illustrative examples of MAO inhibitors include reported inhibitors of the MAO-A isoform, which preferentially deaminates 5-hydroxytryptamine (serotonin) (5-HT) and norepinephrine (NE), and/or the MAO-B isoform, which preferentially deaminates phenylethylamine (PEA) and benzylamine (both MAO-A and MAO-B metabolize Dopamine (DA)). In various embodiments, MAO inhibitors may be irreversible or reversible (e.g., reversible inhibitors of MAO-A (RIMA)), and may have varying potencies against MAO-A and/or MAO-B (e.g., non-selective dual inhibitors or isoform-selective inhibitors).

Certain embodiments include MAO inhibitors that may be selected from the group consisting of: b-carbolines class of inhibitors, tryptoline, pinoline, selegiline, phenelzine, tranylcypromine, hydroxymethyl-beta-carboline, oclobemide, harmane, harmine, luteolin, quercetin, flavonols and flavones and flavonoids, amiflamine, brofaromine, clorgyline, alpha-ethyltryptamine, iproclozide, iproniazid, isocarboxazid, mebanazine, moclobemide, nialamide, pargyline, pheniprazine, pirlindole, safrazine, toloxatone, and tranlcypromine.

In one embodiment, a psychedelic compound of the invention may include psilocybin or psilocin derived from a “psilocybin mushroom,” which includes a polyphyletic, informal group of fungi that contain psilocybin, psilocin or both within their biomass, typically within their fruiting bodies, resulting in their activation of a psychedelic reaction in a subject. Biological genera containing psilocybin mushrooms within the scope of the invention include: Copelandia, Gymnopilus, Inocybe, Panaeolus, Pholiotina, Pluteus, and Psilocybe.

In certain embodiments, the invention may include a composition configured to be administered to a subject in need thereof, containing a psychedelic compound, and preferably psilocybin and/or psilocin and a secondary agent that prevents the metabolic processing of the psychedelic compound, or the that prevent the physiological effect of the psychedelic compound. In a preferred embodiment, a the secondary agent may include a UGT inhibitor and/or a MAOI. In this embodiment, the composition of the invention may be used to treat a serotonin receptor related disease or condition in a subject.

As used herein, a serotonin receptor related disease or condition may be selected from the group consisting of: schizophrenia, addiction, depression, obsessive compulsive disorder (OCD), cluster headaches, dementia, Alzheimer's disease, paralysis, attention deficit-hyperactivity disorder (ADHD), eating disorders, post-traumatic stress disorder (PTSD), anxiety, fear memories, maladaptive stress responses and autism spectrum disorders among others.

Certain embodiments of a composition of the invention may include one or more combinations provided in Table 1 below:

TABLE 1
Exemplary combinations of compositions of the invention containing
a: psychedelic compound (s); first secondary agent; and 3)
additional secondary agent in one embodiment thereof.

| Psychedelic | Secondary Agent | Additional |
|---|---|---|
| Compound | UGT Inhibitor | Secondary Agent |
| psilocybin mushroom | UGT inhibitor | |
| or extract | | |
| psilocybin mushroom | UGT1A9 inhibitor | |
| or extract | | |
| psilocybin mushroom | UGT1A10 inhibitor | |
| or extract | | |
| psilocybin mushroom | UGT1A9 inhibitor | UGT1A10 inhibitor |
| or extract | | |
| psilocybin mushroom | UGT inhibitor | MAOI |
| or extract | | |
| Psilocybin | UGT inhibitor | |
| Psilocybin | UGT1A9 inhibitor | |
| Psilocybin | UGT1A10 inhibitor | |
| Psilocybin | UGT1A9 inhibitor | UGT1A10 inhibitor |
| Psilocybin | UGT inhibitor | MAOI |
| Psilocin | UGT inhibitor | |
| Psilocin | UGT1A9 inhibitor | |
| Psilocin | UGT1A10 inhibitor | |
| Psilocin | UGT1A9 inhibitor | UGT1A10 inhibitor |
| Psilocin | UGT inhibitor | MAOI |
| Psilocybin and Psilocin | UGT inhibitor | |
| Psilocybin and Psilocin | UGT1A9 inhibitor | |
| Psilocybin and Psilocin | UGT1A10 inhibitor | |
| Psilocybin and Psilocin | UGT1A9 inhibitor | UGT1A10 inhibitor |
| Psilocybin and Psilocin | UGT inhibitor | MAOI |

As used herein, the term “glycosyltransferase” or “UDP glucuronosyltransferase,” or “UGT” enzymes refers to a family of enzymes with at least 19 identified human subtypes (enzyme isoforms). The subtypes UGT1A9 and UTG1A10 are isoforms within the family of human UGT enzymes. Certain embodiments include UGT inhibitors, selected from the group consisting of: dapagliflozin, canagliflozin, probenecid, sulfinpyrazone, lamotrigine, atazanavir, gemfibrozil, indinavir, valproic acid. UGT inhibitors may further include a pharmaceutically acceptable salt, or a pharmaceutically acceptable carrier thereof. Other inhibitors of UGT enzymes are known to one of skill in the art and reported in the literature, such as, but not limited to: p-(di-n-propyl sulphamyl)-benzoic acid (probenecid), 5,7-dihydroxyflavone (chrysin), difluorophenyl)-2-hydroxybenzoic acid (diflunisal), 2-((2,3-dimethylphenyl)amino)benzoic acid (mefenamic acid), (2R,3R)-3,5,7-trihydroxy-2-[(2R,3R)-3-(4-hydroxy-3-methoxyphenyl)-2-(hydroxymethyl)-2,3-dihydrobenzo[b][1,4]dioxin-6-yl]chroman-4-one (silibinin), 5,6,7,8-tetramethoxy-2-(4-methoxyphenyl)-4H-1-benzopyran-4-one 5,6,7,8,4′-pentamethoxyflavone (tangeretin), 1-acetyl-4-(4-{[(2R,4S)-2-(2,4-dichlorophenyl)-2-(1H-imidazol-1-ylmethyl)-1,3 -dioxolan-4-yl]methoxy}phenyl)piperazine (ketoconazole), 1-(butan-2-yl)-4-{4-[4-(4-{[2R,4 S)-2-(2,4-dichlorophenyl)-2-(1H-1,2,4-triazol-1-ylmethyl)-1,3-dioxolan-4- yl]methoxy}phenyl)piperazin-1-yl]phenyl}-4,5-dihydro-1H-1,2,4-triazol-5-one (itraconazole), 5-thiazolylmethyl ((alphaS)-alpha-((1S,3S-1-hydroxy-3-((2S)-2-(3-((2-isopropyl-4-thiazolyl)methyl)-3-methylureido)-3-methylbutyramido)-4-phenylbutyl)phenethyl)carbamate (ritonavir), 5-((3,4-dimethoxyphenethyl)methylamino)-2-(3,4-dimethoxyphenyl)-2-isopropylvaleronitrile (verapamil), (+)-dipentene (D-limonene), 2′,4′,5′,7′-tetrabromo-4,5,6,7-tetrachlorofluorescein (cyanosine), bilirubin, (5α,14β,18R)-17-(cyclopropylmethyl)-18-[(1S)-1-hydroxy-1,2,2-trimethylpropyl]-6-methoxy-18,19-dihydro-4,5-epoxy-6,14-ethenomorphinan-3-ol (buprenorphine), (22R,25R)-3β-hydroxy-5α-spirostan-12-one (hecogenin), 1-napthol, 2-{[3-(trifluoromethyl)phenyl]amino}pyridine-3-carboxylic acid (niflumic acid), or 2-(2-((2,6-dichlorophenyl)amino)phenyl)acetic acid (diclofenac).

In certain embodiments, the composition of the invention is provided or used in a “neat” form. As used herein, the term “neat” with respect to a composition of the invention is one that is pure, essentially pure, or substantially pure, generally being referred to as “isolated”. A neat or isolated composition may be identifiable as consisting of a homogeneous chemical or molecular structure, or essentially or nearly so. A neat compound may be obtained through purification procedures to select the molecule out of a mixture of different molecules, through isolation and may be produced, for examples, by biological, synthetic biological, or chemical synthetic methods. The use of the term isolate or neat, includes reference to chemicals, molecules, biological molecules, and any single component system or nearly single component system. The combination of two or more neat or isolated compounds herein is referred to as an admixture.

In certain embodiments, it is preferred to provide or use a psilocin compound that is isolated as described herein. In certain embodiments, isolated psychedelic compounds provide more certainty in dose, free of contaminants or toxins brought by the psilocybin metabolism or growth substrate. For example, in certain embodiments, the provision or use of a psilocybin mushroom, or portion thereof, for treatment of a mental disorder, such as PTSD, has certain less-preferred aspects, including: contamination, or potential contamination, of the psilocybin mushroom with unexpected agents that interfere with a therapeutic use, contamination with toxins or toxins synthesized by the mushrooms, and dose uncertainty. Contaminants and toxins may potentially be within the mushroom, the soil or substrate in which the mushroom grows, or chemicals and the like applied purposely or in course of growth without intention. Dose uncertainty includes uncertainty in the amount of one or more psychedelic compounds in any given mushroom or portion thereof, uncertainty in the amount of other compounds that may affect potency of the psychedelic compound(s) and in ratios of the components of the mushroom having a biological, molecular, or therapeutic activity. In an example, the amounts of psilocybin, psilocin, and the one or more MAO-A inhibitors and ratios thereof, differ between psilocybin mushrooms and even within different parts of a single fruiting body.

Certain embodiments provided herein include one or more isolated compounds useful for treatment of a disease or condition in a subject. Certain embodiments herein, provide one or more mixtures or formulations made from isolated compounds, generally referred to herein as an admixture, such that they are comprised of defined components or comprised of isolated components each in defined amounts or in defined ratios in the formulation (i.e., a defined mixture or defined formulation). Certain embodiments provide a pharmaceutical system or kit having one or more neat compounds, one or more defined formulations, or a combination of one or more isolated compounds, separately, in separate containers or containment, or in an admixture, and one or more defined formulations. Certain embodiments, provide a combination of a biomass or extract or isolate of a biomass, preferably of a psilocybin mushrooms and an isolated compound or admixture of neat compounds herein. Biomass includes a plant or fungal material, and preferably a psilocybin mushroom. Certain embodiments improve on the use of psychedelic mushrooms for therapy of a medical condition or use for altering a biological pathway or response for reasons that include an enhanced certainty in the amount or dose of an isolated compound, combinations of separately provided isolated compounds, admixtures, or a formulation of isolated components provided by said compounds or components.

Certain embodiments herein, disclose the use of individual compounds or defined mixtures thereof as improvements in treatment of serotonin receptor related diseases or conditions, including PTSD, and other described herein. Certain preferred embodiments include methods of treating a serotonin receptor related disease or condition in a subject in need thereof by providing a therapeutically effective amount of a composition of the invention to a subject in need thereof. In a preferred embodiment, a composition of the invention may preferably include a pharmaceutical composition comprising:
a psychedelic compound;
at least one secondary agent; and
and at least one pharmaceutically acceptable carrier.

In alternative embodiments, a composition of the invention may preferably include a pharmaceutical composition comprising:
a psychedelic compound, and preferably psilocybin and/or psilocin or a combination of both;
at least one UGT inhibitor; and
optionally, an MAO inhibitor;
and at least one pharmaceutically acceptable carrier.

In alternative embodiments, a composition of the invention may preferably include a pharmaceutical composition comprising:
a psychedelic compound, and preferably psilocybin and/or psilocin;
at least one UGT inhibitor, and at least one MAO inhibitor;
and at least one pharmaceutically acceptable carrier.

In alternative embodiments, a composition of the invention may preferably include a pharmaceutical composition comprising:
a psychedelic compound, and preferably psilocybin and/or psilocin;
one or more UGT inhibitor, one or more MAO inhibitor or a combination of the same;
and at least one pharmaceutically acceptable carrier.

Administration in vivo of a composition of the invention can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell/receptor being treated, and the subject being treated. Single or multiple administrations can be carried out with the dose level and pattern being selected by the treating physician.

Certain embodiments provide a psychedelic compound, such a psilocin and a MAOI. The

MAOI is administered at the time of the psychedelic compound or prior to the psychedelic compound (as separate components or in combination such as an admixture). Formulations for the MAOI include embodiments of delivery/formulations for: oral, intranasal, tablet, coated tablet, solution, isotonic solution, enteric coated, spray, nasal spray, metered nasal device, dermal patch, injection, systemic injection (e.g., i.v. administration) and other routes of administration such as a suppository (rectal, vaginal). In certain preferred embodiments, the psychedelic compound is provided in formulation for intranasal administration.

In general, a suitable dose of the active compound is in the range of about 100 μg to about 250 mg per kilogram body weight of the subject per day. In general, the dosage, i.e., the therapeutically effective amount, ranges from 1 μg to 10 g/kg and often ranges from 10 μg to 1 g/kg or 10 μg to 200 mg/kg body weight of the subject being treated, per day. Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound, for example a composition of the invention, and preferably a serotonin receptor agonist, such as psilocin, calculated to produce the desired therapeutic effect in association with the required pharmaceutical carrier and a secondary agent. The specification for the dosage unit forms of the invention are dictated by and directly dependent on (a) the unique characteristics of the composition and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a subject in practicing the present invention.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of serotonin receptor agonists, such as a psilocin are well-known in the relevant art and would be understood to be encompassed by the skilled artisan once provided the teachings disclosed herein.

The amount of a composition of the invention, and preferably a serotonin receptor agonist, such as psilocin and one or more secondary agents, administered will be dependent on the subject being treated, the severity of the disorder or condition, the rate of administration, the disposition of the compound and the discretion of the prescribing physician. However, an effective dosage is typically in the range of about 0.001 to about 100 mg per kg body weight per day, preferably about 0.01 to about 35 mg/kg/day, in single or divided doses. For a 70 kg human, this would amount to about 0.07 to about 7000 mg/day, preferably about 0.7 to about 2500 mg/day. In some instances, dosage levels below the lower limit of the aforesaid range may be more than adequate, while in other cases still larger doses may be used without causing any harmful side effect, with such larger doses typically divided into several smaller doses for administration throughout the day. In one preferred embodiment, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.1 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

Administration of a composition of the invention may be effected by any method that enables delivery of the compositions to the site of action. These methods include oral routes, intraduodenal routes, parenteral injection (including intravenous, subcutaneous, intramuscular, intravascular or infusion), topical, and rectal administration.

As noted above, certain compositions of the invention provide a psychedelic agent and a UGT enzyme inhibitor, and/or a MAO enzyme inhibitor, or a combination of the same. Each of these agents are formulated for separate provision, such as in a system or kit of the present invention for a route of administration to include: oral, nasal, dermal patch injection, systemic injection (e.g., iv administration) and other routes of administration such as suppository (rectal, vaginal) as described herein. In certain embodiments, multiple routes of administration may be considered for each separate element of a composition of the invention, such as a psychedelic compound and a secondary agent, which are also generally referred to as an inhibitor, such as a UGT inhibitor, MAO inhibitor, or both. In certain other embodiments, multiple temporal administration protocols may be considered for each separate element of a composition of the invention, such as a psychedelic compound and a secondary agent or inhibitor, such as a UGT inhibitor, MAO inhibitor, or both may be sequentially administered in a single or separate doses which may further be subject to a time-release mechanism.

For example, the invention include a transdermal delivery system. In this embodiment psychedelic compound may be impregnated into in a transdermal patch, and preferably one, or a plurality of separate a time-release transdermal patches configured to release a quantity of the psychedelic compound in a non-intoxicating or sub-intoxicating experience inducing amount. As used herein, the term “non-intoxicating amount or sub-intoxicating amount” means a therapeutically effective amount that treats a serotonin related disease or condition, but that does not induce psychedelia in a subject.

In another embodiment, the invention may further include a transdermal composition having a pharmaceutically effective amount of a composition of the invention for delivery to the bloodstream of a user. This transdermal composition may include a pharmaceutically acceptable carrier, such as an excipient and at least one compositions of the invention capable of diffusing from the composition into the bloodstream of the user. In a preferred embodiment, a pharmaceutically acceptable excipient may be used to create a transdermal dosage form selected from the group consisting of: gels, ointments, cataplasms, poultices, pastes, creams, lotions, plasters and jellies.

The transdermal composition may further include one or more surfactants. In one preferred embodiment, the surfactant may include a surfactant-lecithin organogel, which may further be present in an amount of between about between about 95% and about 98% w/w. In an alternative embodiment, a surfactant-lecithin organogel comprises lecithin and PPG-2 myristyl ether propionate and/or high molecular weight polyacrylic acid polymers. The transdermal composition may further include a quantity of isopropyl myristate. The invention may further include transdermal composition having one or more permeation enhancers to facilitate transfer of the compositions of the invention across a dermal layer. In a preferred embodiment, a permeation enhancer may include one or more of the following: propylene glycol monolaurate, diethylene glycol monoethyl ether, an oleoyl macrogolglyceride, a caprylocaproyl macrogolglyceride, and an oleyl alcohol.

In another embodiment thereof, the present invention, relates to an transdermal patch delivery apparatus which includes a composition of the invention; an impermeable backing; and a rate-controlling microporous membrane. The backing and the membrane define a cavity there between, and the compositions of the invention is disposed within this cavity. In certain embodiments, the each components of the pharmaceutical composition may be coupled with said transdermal delivery device and further associated with pharmaceutically acceptable carriers, each having a different rate of time-release to provide a controlled sequential transdermal administration of the pharmaceutical composition(s) of the invention.

The composition of the invention can include an aqueous medium, which can contain a water- and oil-miscible solvent, or other appropriate aqueous solution of solvent. The invention may further include transdermal composition having one or more permeation enhancers to facilitate transfer of the compositions of the invention across a dermal layer. Depending on the nature of the chosen solvent, the solvent can also act as the permeation enhancer, or a separate permeation enhancer having the desired miscibility can be added to the composition of the invention. In a preferred embodiment, a permeation enhancer may include one or more of the following: propylene glycol monolaurate, diethylene glycol monoethyl ether, an oleoyl macrogolglyceride, a caprylocaproyl macrogolglyceride, and an oleyl alcohol.

By way of understanding, a transdermal patch of the invention releases a psychedelic compound, preferably psilocybin or psilocin, over the duration of the life of the patch on the skin of a person such that the person receives an amount of the compound over time such that the dose does not induce psychedelia in the person. In certain embodiments, the psychedelic compound is a psilocybin, a psilocin, a pharmaceutically acceptable salt thereof, or a combination thereof In certain embodiments, the psychedelic compound is a psilocin or a pharmaceutically acceptable salt thereof. By way of understanding, a transdermal patch of the invention releases a psychedelic compound, preferably psilocybin or psilocin and a quantity of an inhibitor of a UGT enzyme, MOA enzyme, or a combination of the same, wherein the amount of the psychedelic compound, such as psilocin is administered continuously and preferably in a time-release manner to generate a therapeutically effective amount to treat a serotonin related disease or disorder. As noted above, the psychedelic compound, preferably psilocybin or psilocin may include a non- or sub-psychedelic dose of a psychedelic compound, that dose also being modulated by the time-release of the one or more inhibitors of the composition that inhibit, for example psilocin metabolism or its physiological effects, such as serotonin levels in a subject, which results in a reduced amount of the psychedelic compound, such as psilocin needed to produce the desired therapeutic effect.

In certain embodiments, one or more psychedelic compounds, such as psilocybin or psilocin, or a combination of the same, are administered prior to administration of the secondary agent, which may preferably include a UGT inhibitor or a MAOI or a combination of the same. In certain additional embodiments, one or more psychedelic compounds, such as psilocybin or psilocin, or a combination of the same, are administered concurrently with the administration of a secondary agent, which may preferably include a UGT inhibitor, a MAOI or a combination of the same.

In alternative embodiments one or more secondary agents of the invention are administered prior to administration of the psychedelic compound, and preferably transdermally as generally describe herein. For example, an inhibitor may be administered by patch for one or several hours; or one, two, three, four, five, six, or seven days (same patch or replaced periodically with a fresh patch and additional dose or amount of inhibitor) and this is done prior to delivery of the psychedelic agent which may be administered by any route available, including those described herein. As shown in Table 2 below, exemplary administration protocols may include:

TABLE 2
Exemplary Administration protocols

| Enhancer | Administration Protocol | Psychedelic Agent |
|---|---|---|
| UGT inhibitor (UGTi) | UGTi administered at same or | psilocin (alt. psilocybin) |
| (optionally include an | similar time to that of the | |
| MAOinhibitor (MAOi)) | psychedelic agent (optionally in | |
| | an admixture, such as a capsule, | |
| | tablet, or in a drink) | |
| UGTi (optionally + MAOi) | UGTi 30 min or more prior to | psilocin (alt. psilocybin) |
| | psychedelic | |
| UGTi (optionally + MAOi) | UGTi one hour or more prior to | psilocin (alt. psilocybin) |
| | psychedelic | |
| UGTi (optionally + MAOi) | UGTi two hours or more prior to | psilocin (alt. psilocybin) |
| | psychedelic | |
| UGTi (optionally + MAOi) | UGTi three hours or more | psilocin (alt. psilocybin) |
| | priorto psychedelic | |
| UGTi (optionally + MAOi) | UGTi six hours or more prior to | psilocin (alt. psilocybin) |
| | psychedelic | |
| UGTi (optionally + MAOi) | UGTi from one to 24 hours prior | psilocin (alt. psilocybin) |
| | to psychedelic | |
| UGTi (optionally + MAOi) | UGTi from 6 to 24 hours prior to | psilocin (alt. psilocybin) |
| | psychedelic | |
| UGTi (optionally + MAOi) | UGTi from 12 to 48 hours | psilocin (alt. psilocybin) |
| | priorto psychedelic | |
| UGTi (optionally + MAOi) | UGTi about one day prior to | psilocin (alt. psilocybin) |
| | psychedelic (optionally with a | |
| | one or more repeated | |
| | administrations of the UGTi) | |
| UGTi (optionally + MAOi) | UGTi about two days prior to | psilocin (alt. psilocybin) |
| | psychedelic (optionally with a | |
| | one or more repeated | |
| | administrations of the UGTi | |
| | withfirst dose about two days | |
| | prior) | |
| UGTi (optionally + MAOi) | UGTi about three days prior to | psilocin (alt. psilocybin) |
| | psychedelic (optionally with a | |
| | one or more repeated | |
| | administrations of the UGTi | |
| | withfirst dose about three days | |
| | prior) | |
| UGTi (optionally + MAOi) | UGTi about four, five, six or | psilocin (alt. psilocybin) |
| | seven days prior to psychedelic | |
| | (optionally with a one or more | |
| | repeated administrations of the | |
| | UGTi with first dose about four, | |
| | five or six days prior, respectively) | |

In certain embodiments, a non- or sub-psychedelic dose of a psychedelic compound, and preferably psilocybin or a psilocin is administered for a time period of days, weeks, months, or years (referred to herein as a continuum treatment) and is effective for treatment of a serotonin related disease or disorder. In certain embodiments, the continuum treatment is combined, in any order, with one or more treatments, optionally one treatment, with a dose of the psychedelic compound effective to induce a therapeutic psychedelic experience, which, in certain embodiments is coupled with one or more physical or mental interventions, such as group or one-on-one counseling.

In certain embodiments, the transdermal patch system, or any delivery system described herein may include a psychedelic compound and one, or a plurality of secondary agents generally referred to a s a first, second or third inhibitors and so on. Non-limiting examples are provided in Table 3 below.

TABLE 3
Example compositions for transdermal or other routes of delivery.

| Psychedelic | First Inhibitor | Second Inhibitor | Third Inhibitor |
|---|---|---|---|
| Psilocybin | UGT inhibitor | | |
| Psilocybin | UGT1A9 inhibitor | | |
| Psilocybin | UGT1A10 inhibitor | | |
| Psilocybin | UGT1A9 inhibitor | UGT1A10 inhibitor | |
| Psilocybin | UGT inhibitor | MAO inhibitor | |
| Psilocybin | UGT inhibitor | MAO-A inhibitor | |
| Psilocybin | UGT1A9 inhibitor | MAO inhibitor | |
| Psilocybin | UGT1A9 inhibitor | MAO-A inhibitor | |
| Psilocybin | UGT1A10 inhibitor | MAO inhibitor | |
| Psilocybin | UGT1A10 inhibitor | MAO-A inhibitor | |
| Psilocybin | UGT1A9 inhibitor | UGT1A10 inhibitor | MAO inhibitor |
| Psilocybin | UGT1A9 inhibitor | UGT1A10 inhibitor | MAO-A inhibitor |
| Psilocybin | MAO inhibitor | | |
| Psilocybin | MAO-A inhibitor | | |
| Psilocin | UGT inhibitor | | |
| Psilocin | UGT1A9 inhibitor | | |
| Psilocin | UGT1A10 inhibitor | | |
| Psilocin | UGT1A9 inhibitor | UGT1A10 inhibitor | |
| Psilocin | UGT inhibitor | MAO inhibitor | |
| Psilocin | UGT inhibitor | MAO-A inhibitor | |
| Psilocin | UGT1A9 inhibitor | MAO inhibitor | |
| Psilocin | UGT1A9 inhibitor | MAO-A inhibitor | |
| Psilocin | UGT1A10 inhibitor | MAO inhibitor | |
| Psilocin | UGT1A10 inhibitor | MAO-A inhibitor | |
| Psilocin | UGT1A9 inhibitor | UGT1A10 inhibitor | MAO inhibitor |

In one example, a therapeutically effective amount of a pharmaceutical composition of the invention may be transdermally delivered, preferably with a transdermal patch, to a subject in need thereof, said composition comprising: a first compound psychedelic compound configured to be administered transdermally; one or more secondary agents, such as an inhibitor of an enzyme or enzyme activity known to metabolize the psychedelic compound in vitro or in vivo. In certain embodiments, the inhibitor is a monoamine oxidase (MAO) inhibitor. In certain embodiments, the inhibitor is a MAO-A or MAO-B inhibitor (referring to the A or B subtype of the MAO family of enzymes). In certain embodiments the inhibitor is a UGT inhibitor and in certain embodiments inhibits UGT1A9, UGT1A10, or both types of enzymes. In certain embodiments, the inhibitor includes an inhibitor of an MAO enzyme, an inhibitor of a UGT enzyme, inhibitors that inhibit both enzymes, or multiple inhibitors one or more of which inhibits a UGT enzyme and/or one or more of which inhibits an MAO enzyme.

Notably, in one embodiment MAO may facilitate the oxidation and degradation of serotonin to 5-hydroxyindole. Generally, in MAO-A has shown a higher affinity to the oxidation and degradation of serotonin to 5-hydroxyindole than MAO-B. Also, MAO-A and MAO-B are implicated in the metabolism of psilocin, namely the oxidation of psilocin to 4-hydroxyindoleacetic acid. As such, as generally described herein, inhibition of MAO may include the MAO-A, MAO-B, or a combination of the same.

Certain embodiments provide a kit for metering a sub-psychedelic dose of a psychedelic compound, such as psilocin, administered to a person through preferably a transdermal device such as a patch, comprising: a psychedelic compound and an inhibitor of an enzymatic degradation of the psychedelic compound, wherein at least the psychedelic compound is included in a formulation for transdermal administration. In this embodiment, the kit may include a composition of the invention in the form of one or more impregnated transdermal patches, a container and instructions for administration.

In certain embodiments of the invention, the transdermal delivery system may administer to a subject in need thereof a dose of a psychedelic compound, and preferably a dose of psilocin that is within a nanodose range, being defined as approximately less than 1 microgram per 1 kg of body weight of the person desirous of or in need of the dose of psilocin; which is effective in association with one or more of secondary agents as described herein. In certain embodiments, the dose of psilocin, in the alternative to nanodose range, is at or below 2 mg per dose or at or below single digit milligrams per kg of the person receiving the administration. In certain embodiments, the treatment of a post-traumatic stress disorder (PTSD), and the psilocin and the inhibitor of psilocin metabolism, such as a UGT inhibitor, are administered in amounts effective to inhibit the psilocin metabolism (by at least 20% of normal population range or by at least 20% for the instant patient (person)) and the psilocin in a quantity to induce a psychedelic experience in the person or to provide an effective treatment of the PTSD.

Certain embodiments of the invention may include different administration regimes of individual compounds or defined mixtures thereof as improvements in treatment of a serotonin-related disease or disorders, as shown in the non-limiting examples provided in Table 4 below

TABLE 4
Exemplary transdermal dosing regimens of one
or more compositions of the invention.

| Inhibitor | Administration Protocol | Psychedelic Agent |
|---|---|---|
| UGT inhibitor (UGTi) | inhibitor(s) administered at same | psilocin |
| and/orMAO inhibitor | or similar time to that of the | (alternatively, psilocybin) |
| (MAOi) | psychedelic agent | |
| UGT inhibitor (UGTi) | inhibitor(s) 30 min or more | psilocin |
| and/orMAO inhibitor | priorto psychedelic | (alternatively, psilocybin) |
| (MAOi) | | |
| UGT inhibitor (UGTi) | inhibitor(s) one hour or | psilocin |
| and/orMAO inhibitor | moreprior to psychedelic | (alternatively, psilocybin) |
| (MAOi) | | |
| UGT inhibitor (UGTi) | inhibitor(s) two hours or | psilocin |
| and/orMAO inhibitor | moreprior to psychedelic | (alternatively, psilocybin) |
| (MAOi) | | |
| UGT inhibitor (UGTi) | inhibitor(s) three hours or | psilocin |
| and/orMAO inhibitor | moreprior to psychedelic | (alternatively, psilocybin) |
| (MAOi) | | |
| UGT inhibitor (UGTi) | inhibitor(s) six hours or | psilocin |
| and/orMAO inhibitor | moreprior to psychedelic | (alternatively, psilocybin) |
| (MAOi) | | |
| UGT inhibitor (UGTi) | inhibitor(s) from one to 24 | psilocin |
| and/orMAO inhibitor | hoursprior to psychedelic | (alternatively, psilocybin) |
| (MAOi) | | |
| UGT inhibitor (UGTi) | inhibitor(s) from 6 to 24 | psilocin |
| and/orMAO inhibitor | hoursprior to psychedelic | (alternatively, psilocybin) |
| (MAOi) | | |
| UGT inhibitor (UGTi) | inhibitor(s) from 12 to 48 | psilocin |
| and/orMAO inhibitor | hoursprior to psychedelic | (alternatively, psilocybin) |
| (MAOi) | | |
| UGT inhibitor (UGTi) | inhibitor(s) about one day prior | psilocin |
| and/orMAO inhibitor | to psychedelic (optionally with | (alternatively, psilocybin) |
| (MAOi) | aone or more repeated | |
| | administrations of the | |
| | inhibitor(s)) | |
| UGT inhibitor (UGTi) | inhibitor(s) about two days prior | psilocin |
| and/orMAO inhibitor | to psychedelic (optionally with a | (alternatively, psilocybin) |
| (MAOi) | one or more repeated | |
| | administrations of the | |
| | inhibitor(s)with first dose about | |
| | two days prior) | |
| UGT inhibitor (UGTi) | inhibitor(s) about three days | psilocin |
| and/orMAO inhibitor | priorto psychedelic (optionally | (alternatively, psilocybin) |
| (MAOi) | with a one or more repeated | |
| | administrations of the | |
| | inhibitor(s)with first dose about | |
| | three days prior) | |

Certain embodiments of the present invention provide compositions, and preferably pharmaceutical compositions, which may be further provided as a kit comprising: a psychedelic compound and an inhibitor of a monoamine oxidase (MAO) enzyme that results in the reduction of MAO activity in a subject, wherein at least the psychedelic compound is included in a formulation for intranasal administration. In certain embodiments, the psychedelic compound and the inhibitor are included, in combination, in a formula for intranasal administration. In certain embodiments, the compound and the inhibitor are provided in separate formulations for administration to the person. In certain embodiments, the formula is an aqueous solution, such as an isotonic solution, for example, an isotonic saline solution. In certain embodiments, the inhibitor is administered prior to the administration of the psychedelic compound.

Certain embodiments of the present invention include compositions comprising:
a psychedelic compound;
at least one secondary agent,
optionally, a pharmaceutically acceptable carrier, and preferably a preservative of the psychedelic compound;
wherein the composition includes one or more pharmaceutically acceptable nasal formulations, preferably isotonic nasal formulations.

Certain embodiments of the present invention include compositions comprising:
a psychedelic compound selected from psylocibin and psilocin, or a combination of both;
at least one secondary agent, selected from a MAOI or UGT inhibitor, or a combination of both
a pharmaceutically acceptable carrier, and preferably a preservative of the psychedelic compound and/or secondary agent;
wherein the composition includes one or more pharmaceutically acceptable nasal formulations, preferably isotonic nasal formulations.

Certain embodiments of the present invention include compositions comprising:
a psychedelic compound selected from psylocibin and psilocin, or a combination of both;
a MAOI
a pharmaceutically acceptable carrier, and preferably a preservative of the psychedelic compound and/or MAOI;
wherein the composition includes one or more pharmaceutically acceptable nasal formulations, preferably isotonic nasal formulations.

In certain embodiments of the present invention, the psychedelic compound, preferably psilocin, and the secondary agent, preferably an inhibitor are in separate containers/formulations, preferably both in pharmaceutically acceptable nasal formulations for intranasal delivery. In certain embodiments, the intranasal formulation includes psilocin and the dose of the psilocin is in the nanodose range, being defined as below 1 microgram per 1 kg of body weight of the person desirous of or in need of the dose of psilocin, which is effective in association with one or more of: 1) the nose to brain delivery which bypasses first pass metabolism including the UGT enzymes which metabolize the majority of administered psilocybin or psilocin; and the inhibition of the MAO, preferably MAO-A, enzymes that are found in the brain and metabolize psilocin, but have reduced metabolic activity as a result of the administration of the inhibitor of the MAO enzyme(s).

In certain embodiments of the present composition of the invention, the dose of psilocin, in the alternative to nanodose range, is at or below 2 mg per dose or at or below single digit milligrams per kg of the person receiving the administration.

In certain embodiments of the present composition of the invention, psilocin and the inhibitor of psilocin metabolism are administered in amounts effective to inhibit the psilocin metabolism (by at least 20% of normal population range or by at least 20% for the instant patient (person)) and the psilocin in a quantity to induce a psychedelic experience in the person or to provide an effective treatment of the PTSD.

Certain embodiments of a psychedelic plus an enzymatic inhibiting agent are provided in

TABLE 5
exemplary composition formulations for intranasal administration.

| Psychedelic | Agent |
|---|---|
| Psilocybin | MAO inhibitor |
| Psilocybin | MAO-A inhibitor |
| Psychedelic mushroom | MAO inhibitor |
| Psychedelic mushroom | MAO-A inhibitor |
| Psilocin | MAO inhibitor |
| Psilocin | MAO-A inhibitor |
| Psilocybin and Psilocin | MAO inhibitor |
| Psilocybin and Psilocin | MAO-A inhibitor |

Certain administration regimens are embodied in Table 6 below.

TABLE 6
exemplary intranasal administration regimes for intranasal administration

| Enhancer | Administration Protocol | Psychedelic Agent |
|---|---|---|
| MAO inhibitor (MAOi) | MAOi administered at same or | psilocin |
| | similar time to that of the | (alternatively, psilocybin) |
| | psychedelic agent (as separate | |
| | components or, optionally, in | |
| | anadmixture, such as a capsule, | |
| | tablet, or in a drink) | |
| MAO inhibitor (MAOi) | MAOi 30 min or more prior to | psilocin |
| | psychedelic | (alternatively, psilocybin) |
| MAO inhibitor (MAOi) | MAOi one hour or more prior to | psilocin |
| | psychedelic | (alternatively, psilocybin) |
| MAO inhibitor (MAOi) | MAOi two hours or more prior to | psilocin |
| | psychedelic | (alternatively, psilocybin) |
| MAO inhibitor (MAOi) | MAOi three hours or more prior | psilocin |
| | to psychedelic | (alternatively, psilocybin) |
| MAO inhibitor (MAOi) | MAOi six hours or more prior to | psilocin |
| | psychedelic | (alternatively, psilocybin) |
| MAO inhibitor (MAOi) | MAOi from one to 24 hours prior | psilocin |
| | to psychedelic | (alternatively, psilocybin) |
| MAO inhibitor (MAOi) | MAOi from 6 to 24 hours prior to | psilocin |
| | psychedelic | (alternatively, psilocybin) |
| MAO inhibitor (MAOi) | MAOi from 12 to 48 hours prior | psilocin |
| | to psychedelic | (alternatively, psilocybin) |
| MAO inhibitor (MAOi) | MAOi about one day prior to | psilocin |
| | psychedelic (optionally with a | (alternatively, psilocybin) |
| | one or more repeated | |
| | administrations of the MAOi) | |
| MAO inhibitor (MAOi) | MAOi about two days prior to | psilocin |
| | psychedelic (optionally with a | (alternatively, psilocybin) |
| | one or more repeated | |
| | administrations of the MAOi with | |
| | first dose about two days prior) | |
| MAO inhibitor (MAOi) | MAOi about three days prior to | psilocin |
| | psychedelic (optionally with a | (alternatively, psilocybin) |
| | one or more repeated | |
| | administrations of the MAOi with | |
| | first dose about three days prior) | |
| MAO inhibitor (MAOi) | MAOi about four, five, six or | psilocin |
| | seven days prior to psychedelic | (alternatively, psilocybin) |
| | (optionally with a one or more | |
| | repeated administrations of the | |
| | MAOi with first dose about four, | |
| | five or six days prior, respectively) | |

Certain embodiments provide compositions having a psychedelics compounds and one or more secondary agents that modulate the activity, or physiological response of the subject to the psychedelic compound. Embodiments of a psychedelic compound, include, for example, a psilocybin, or a psilocin compound. Embodiments of secondary agents, include, for example, a compounds that affects the absorption, processing, metabolism, of the psychedelic compound, and in certain embodiments, the psilocybin or psilocin compound in a biological system. Certain embodied secondary agents include inhibitor compounds which inhibits the metabolic breakdown the an agent in a biological system into metabolites, including the breakdown to metabolites which do not express the activity or directly modulate of the initial psychedelic agent such as the psilocin or psilocybin. Certain exemplary embodiments of the present invention provide a description of selected biological activities, processes and responses, certain embodiments of which are as set forth by way of example in Table 5.

TABLE 5
Exemplary biological activities, processes and responses
of one or more compositions of the invention.

| Item | Agent | Action |
|---|---|---|
| Psilocybin and psilocin are | Psilocin is estimated to be more | Binding of psilocin with 5- |
| psychedelic compounds | active in psychedelic effect per | HT2Areceptors in the brain is |
| | unit dose, 10X or more estimated | correlated with psychedelic |
| | | response to psychedelic |
| | | mushroom consumption. |
| Psilocin | 5-HT2A | Associated with binding with |
| | | the 5-HT2A receptor found in |
| | | various brain regions and in |
| | | nerve synapses (alternatively |
| | | known as the serotonin receptor 2A). |
| | Increases dopamine levels or | |
| | activity in | |
| Psilocybin (PY) is metabolized | Alkaline phosphatase enzyme | 50% of psilocybin is |
| topsilocin (PI) | (family), alternatively heat, | not absorbed by human |
| | light, low pH | |
| Psilocin is metabolized to | UDP-glucuronosyltransferase | There are least 19 human UGT |
| psilocin-O-glucuronide (PI-O) | (UGT) | subtypes |
| Psilocin is metabolized to | UGT1A10 (UTG subtype) | Particularly active in the |
| psilocin-O-glucuronide (PI-O) | | intestine, liver |
| Psilocin is metabolized to | UGT1A9 (UTG subtype) | Particularly active in the |
| psilocin-O-glucuronide (PI-O) | | circulation (blood) |
| Psilocin metabolic processing | About 80% of administered | Administration of an amount |
| | psilocin is broken down by | of psilocin results in about |
| | UGT1A10, UGT1A9, or the | 20%, or less, of the psilocin |
| | combination | reaching the brain |
| Psilocin is metabolized to | | Considered non-psychoactive |
| 4-hydroxyindol-3-yl- | | in certain embodiments herein. |
| acetaldehyde | | |

Certain embodiments provide a pharmaceutical composition, comprising: a neat psilocybin and a neat inhibitor of the biological metabolism of the psilocybin in a biological system. As noted herein, the administration of psilocybin to a subject results in about 50% of compound being eliminated from the body without absorption (e.g., absorption in the mouth, stomach, intestine, liver, etc.). Certain additional embodiments provide points of measurement or endpoints for use of one or more composition of the invention in a therapeutic protocol, for example. Certain embodiments utilize a measurement of the blood or plasma level of a psychedelic compound, such as psilocin. This measure may include the absolute or relative concentration of a psychedelic compound such a psilocin concentration in a biological sample provided by a subject. Examples of a “biological sample” may include saliva, tears, sweat, urine, tissue, plasma, blood, cerebral spinal fluid, other bodily fluid; and the like

As further noted above, approximately 5% of the psilocin consumed or metabolized from consumed psilocybin reaches the brain in a typical subject. As a result, a large dose of psilocybin, in neat form, or as part of a psylocibin mushroom biomass, is needed compared to the amount of active metabolite in brain synapses, receptor binding, 5-HT2a receptor binding. As described in certain embodiments herein compositions and methods of use thereof that change the ratio of amount of psychedelic compound administered, enhancing said efficiency of compounds use including using a lower amount administered, enhancing the efficacy of psychedelic use (amount; time of effect; time to onset; duration; intensity of experience (e.g., as reported by the subject or patient); area under the curve of time and intensity of experience; concentration in blood, plasma, tears, saliva, cerebral spinal fluid, other bodily fluid; and the like.

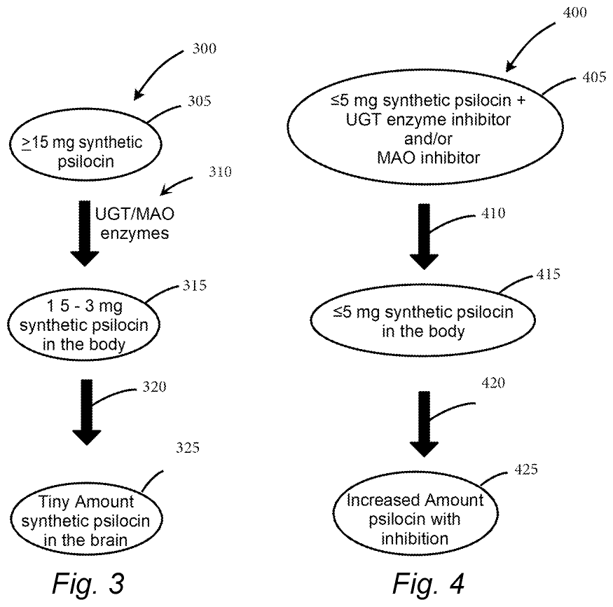

As shown in FIG. 3, a pathway of administration (300) of a therapeutically effective amount of an exemplary psychedelic composition having an example quantity of a synthetic psilocin in an example amount of 15 mg or more (305). The figure shows that enzymes including UGT and MAO type enzymes act on the administered psilocin, eliminating the psilocin by conversion to metabolites through enzymatic action (310). The remaining psilocin (that escaped metabolism to metabolites via enzymatic action) (310) is distributed throughout the body and the total amount remaining after enzymatic action, for example, is approximately one-fifth to one-thirtieth of that administered (315). A result of the metabolic processing is that a small amount of the administered psilocin reaches the brain (320) and even less reaches the 5-HT2A receptor which is a local of action for the psilocin for eliciting an agonistic activation of the receptor and subsequent release of dopamine, and/or an effective therapeutic treatment of a disorder such as PTSD (325) as generally described herein.

FIG. 4, further shows an administration scenario (400) for administration of a synthetic psilocin administration in a reduced amount as one or more UGT or MAO inhibitor compounds reduce the metabolic processing of the administered psilocin (405). In the example shown, 5 mg or less of synthetic psilocin is administered and the inhibitors are administered concurrent with the psilocin, or the inhibitors are administered prior to the psilocin, to achieve a target level of inhibition in the subject before administration of the psilocin (415). The synthetic psilocin distributes throughout the body (420) and interacts with the 5-HT2A receptors with effect of eliciting an agonistic activation of the receptor and subsequent release of dopamine, and/or an effective therapeutic treatment of a disorder such as PTSD (425). The use of the UGT, MAO, or both inhibitors to reduce metabolic processing of the administered psilocin (310) allows for the use of much smaller, therapeutically effective amount or doses of psilocin to achieve a particular experience intensity, time, effect and/or treatment outcome because the end result of the system (400) is that a similar level of 5-HT2A binding with psilocin occurs even with a much smaller administered dose of synthetic psilocin (425 and 405).

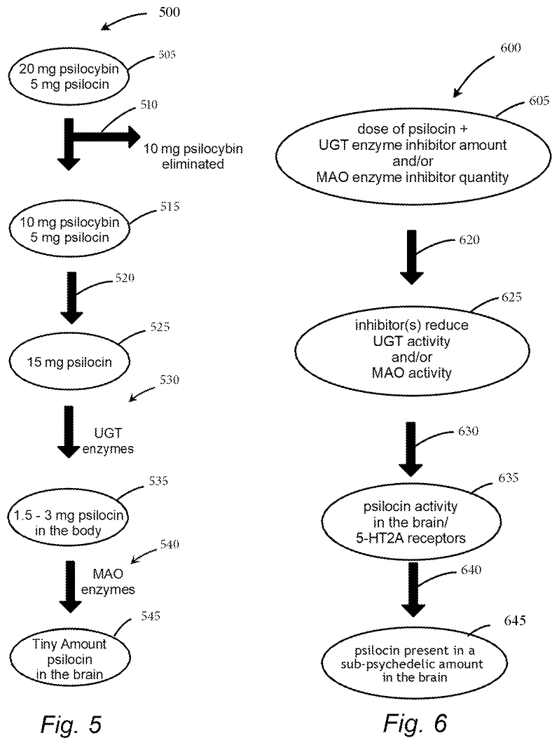

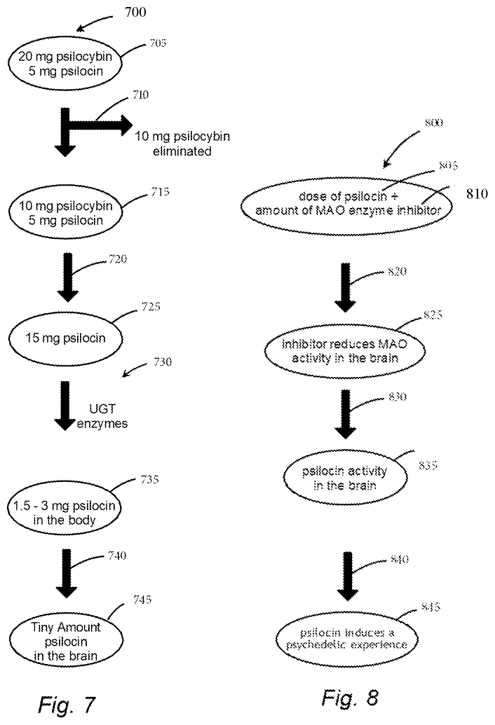

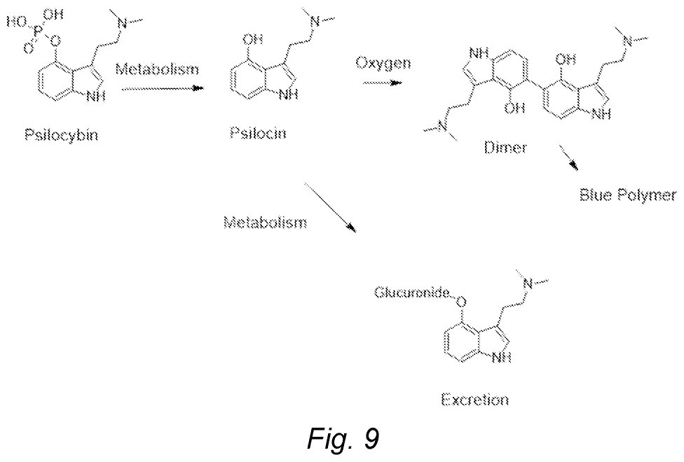

As shown in FIG. 5, an orally administered pathway (500) of an amount of a an amount of psilocybin and/or psilocin (505) for inducing a psychedelic experience in a person desirous thereof, or as a treatment of a serotonin related disease of condition in a person in need thereof. The figure shows that enzymes including alkaline phosphatase (510) and UGT (530) and MAO (540) act on the administered psychedelics resulting in a limited amount of psilocin being distributed throughout the body (535) and being present in the brain/synapses/5-HT2A receptors (the proposed site of psilocin action being the 5-HT2A receptors in the brain or brain synapses, without being bound to mechanism) (545).

FIG. 6 shows a an administration scheme (600) for transdermal administration of a composition of the invention, containing a dose of psilocin (605) and an amount of a UGT enzyme inhibitor and/or a quantity of an MAO inhibitor (605), wherein the MAO inhibitor and/or the UGT inhibitor reduces the metabolism or break down of the psilocin (625) in the body of a subject, allowing migration of psilocin to the brain (630), and proposed binding with 5-HT2A receptors (635), and modulation of a symptom of a disorder (645) or as a biomarker indicative of treatment of a mental disorder in a person in need of such treatment (645).

As shown in FIG. 5, an orally administered pathway (700) of an amount of psilocybin and/or psilocin (705) for inducing a psychedelic experience in a person desirous thereof, or as a treatment of a serotonin related disease of condition in a person in need thereof. The figure shows that enzymes including alkaline phosphatase (710) and UGT (730) and MAO (740) act on the administered psychedelics resulting in a limited amount of psilocin being distributed throughout the body (735) and being present in the brain/synapses/5-HT2A receptors (the proposed site of psilocin action being the 5-HT2A receptors in the brain or brain synapses, without being bound to mechanism) (745).

FIG. 8 shows an pathway (800) for intranasal administration of a dose of a psychedelic compound, in this case psilocin (805) and an amount of an MAO enzyme inhibitor (805) (intranasal or other route of administration, 810); wherein the MAO inhibitor reduces physiological effects of psilocin (825), namely the subjects level of serotonin generated in response to activation of 5-HT2A receptors through the inhibition of monoamine oxidase, and migration of psilocin to the brain (830), proposed binding with 5-HT2A receptors (840), and induction of a psychedelic experience (845) which is a biomarker or indicative of treatment of a serotonin-related disease or disorder in a person in need of such treatment.

As used herein, a “psychedelic compound” or “psychedelic agent” includes serotonin receptor agonists. As used herein, a “serotonin receptor agonists” means a substance, and preferably a compound of the invention, having the function of acting on a serotonin receptor, and includes, for example, a 5-HT2A, 5-HT2C and 5-HT1A 5-HT2A receptor agonist. As used herein, an “agonist” means a substance, and preferably a compound of the invention, having the function of binding/activating to a receptor or to produce a biological response.

In a preferred embodiment, a psychedelic compound may be a serotonin receptor agonists selected from the group consisting of: psilocybin, and psilocin. In a preferred embodiment a psychedelic compound may include “psilocybin” and/or “psilocin” as shown in FIG. 3, including all analogs, racemic forms, enantiomers and the like. Notably, as psilocybin is metabolized in a subject to psilocin, as used herein the administration of psilocybin to a subject also includes psilocybin, its metabolized form psilocin, or a combination of the same.

In certain embodiments, psilocybin or psilocin may be isolated and purified from a psychedelic mushroom. In other embodiment, psilocybin or psilocin may be part of the biomass of a psychedelic mushroom, or an extract of a psychedelic mushroom. As used herein, a compound is referred to as “isolated” or “purified” when it has been separated from at least one component with which it is naturally associated. For example, a compound, such as psilocin can be considered isolated if it is separated from contaminants including components of, for example a psychedelic mushroom extract or biomass, or other components of in vitro synthesis. Isolated molecules can be either prepared synthetically or purified from their natural environment. Standard quantification methodologies known in the art can be employed to obtain and isolate the molecules of the invention. An isolated or purified compound of composition of the invention may include a compositions containing at least 25%, 50%, 60% 70%, 80%, 90%, between 91%-99% or between 99% and 100% of the subject compound(s).

As noted above, certain embodiments provide a pharmaceutical compositions, kit, and methods of treatment, comprising: a psychedelic compound; and an enzyme or compounds known to metabolize the psychedelic compound, wherein the components of the compositions are synthetic, or a mixture of synthetic and isolated naturally occurring compounds. Certain embodiments provide synthetic compounds and combinations for benefits (including avoiding issues or problems with natural-sources products) that encompass the following: 1) harvesting of psychedelic mushrooms for use in products and treatments potentially brings contaminants, confounding factors, toxins, and the like into the system; 2) isolation and purification protocols potentially introduce new confounding factors such as resins from purification columns and contaminants from the resins and reagents; 3) isolation and purification protocols potentially result in breakdown of the compound of interest which contaminates the system, such as through application of heat, pH, and other parameters; and 4) isolation and purification from natural sources, such as plants and fungi, may lead to unsustainable harvesting of the biomass and disruption of sensitive ecosystems.

As such, in further preferred embodiments, psychedelic mushroom may be generated in vitro. Exemplary methods of in vitro synthesis of psilocybin or psilocin are generally disclosed by Shirota, et al., (2003). Concise Large-Scale Synthesis of Psilocin and Psilocybin, Principal Hallucinogenic Constituents of “Magic Mushroom”. Journal of natural products. 66. 885-7. Such synthetic manufacturing methods to produce psilocybin or psilocin are specifically incorporated herein by reference) Additional embodiment may include a psychedelic compound, such as psilocybin or psilocin that are produce in vivo, for example through a yeast or bacterial production system, or a bioreactor. Alternative embodiment may include a combination of in vivo or in vitro synthesis, for example where in precursor tryptamine compositions are generated in an in vivo production system, and further chemically modified in vitro to produce a purified psychedelic compound such as such as psilocybin.

In some embodiments, the pharmacological mechanism of 5-HT2A activity and the release of native serotonin, dopamine and norepinephrine, are accomplished with a single active pharmaceutical composition that may include a serotonin receptor agonists, such as psilocybin or psilocin and a secondary agent, which may preferably include an inhibitor of the metabolism of psilocybin or psilocin, such as a UGT inhibitor, and optionally another secondary agent such as an MAOI. It is the novel compilation of these mechanisms, for reasons stated above, that is useful in therapy for improved outcomes.

In one embodiment, a psychedelic compound may include a tryptamine compound. Tryptamines are naturally occurring monoamine alkaloids derived from tryptophan, from which the name is derived. Analogs within the tryptamine family contain substitutions at the indole ring structure and the amine group. This family of compounds contains psychotropically active members, including N,-N-dimethyltryptophan (DMT), 5-methoxy-N,N-dimethyltryptamine (5-MeO-DMT), 4-hydroxy dimethyl-tryptophan (psilocin) and its 4-O-phosphate ester, psilocybin. Psilocin may act as an agonist on 5HT1A, 5HT2A, and 5HT2C receptors.

The invention encompasses methods of preventing or treating (e.g., alleviating one or more symptoms of) medical conditions through use of one or more of the disclosed composition of the invention, preferably in the form of a pharmaceutical compositions. The methods comprise administering a therapeutically effective amount of a composition of the invention to a patient in need, preferably a psychedelic composition, such as a serotonin receptor agonist like psilocybin or psilocin, and one or more secondary agents, such as a UGT inhibitor or a MAOI and a pharmaceutically acceptable carrier, or a pharmaceutically acceptable salt therewith. The compositions of the invention can also be used for prophylactic therapy.

The “therapeutically effective amount” for the treatment of a subject afflicted with a disorder ameliorated by the described therapy is an amount that causes amelioration of the disorder being treated or protects against a risk associated with the disorder, and in particular a serotonin receptor related disease or condition. For example, for schizophrenia, a therapeutically effective amount is an amount which causes a significant reduction in psychopathology as determined by clinical improvement; for depression, a therapeutically effective amount is an amount that leads to stabilization and remission of symptoms as measured by the Patient Health Questonnaire-9; for OCD, a therapeutically effective amount is an amount that leads to stabilization and remission of symptoms as measured by the Yale-Brown Obsessive Compulsive Scale; for ADHD, a therapeutically effective amount is an amount that leads to stabilization and remission of symptoms as measured by either the ADHD Rating Scale V or ADHD Self-Report Scale; for eating disorders, a therapeutically effective amount is an amount that leads to stabilization and remission of symptoms as measured by the Eating Disorder Examination Questionnaire; for autism spectrum disorders a therapeutically effective amount is an amount that leads to stabilization and remission of symptoms as measured by physicians' assessment; for PTSD a therapeutically effective amount is an amount that leads to stabilization and remission of symptoms as measured by the Clinician-Administered PTSD Scale for DSM-5; for anxiety, a therapeutically effective amount is an amount that leads to stabilization and remission of symptoms as measured by the General Anxiety Disorder-7; for addiction, a therapeutically effective amount is an amount that leads to stabilization and remission of symptoms as measured by physicians' assessment; for cluster headaches, a therapeutically effective amount is an amount that leads to stabilization and remission of symptoms as measured by the Cluster Headache Severity Scale (CHSS); for dementia, a therapeutically effective amount is an amount that leads to stabilization and remission of symptoms as measured by the Dementia Rating Scale (DRS); for Alzheimer's disease, a therapeutically effective amount is an amount that leads to stabilization and remission of symptoms as measured by the Alzheimer's Disease Assessment Scale-Cognitive Subscale (ADAS-Cog); for paralysis, a therapeutically effective amount is an amount that leads to stabilization and remission of symptoms as measured by physicians' assessment.

As used herein, “treating” or “treatment” describes the management and care of a patient for the purpose of combating a disease, condition, or disorder, and in particular serotonin receptor related disease or condition and includes the administration of a compound or pharmaceutical compositions of the present invention to prevent the onset of the symptoms or complications, alleviating the symptoms or complications, or eliminating the disease, condition or disorder. More specifically, “treating” includes reversing, attenuating, alleviating, minimizing, suppressing or halting at least one deleterious symptom or effect of a disease (disorder) state, disease progression, or other abnormal condition, and in particular a serotonin receptor related disease or condition. Treatment is continued as long as symptoms and/or pathology ameliorate. The term “beneficial” or “improved outcomes” as used herein in the context of treating a condition, refers to extended relieve of symptoms (duration) and/or a more significant reduction of symptoms (magnitude), and in particular with respect to a serotonin receptor related disease or disorder. Exemplary serotonin receptor related diseases or disorders can include schizophrenia, depression, obsessive compulsive disorder, attention deficit-hyperactivity disorders, eating disorders, autism spectrum disorders, PTSD and anxiety as discussed above. The phrase “improved outcomes” means an extended relieve of symptoms (duration) and/or a more significant reduction of symptoms (magnitude).

As used herein, a “serotonin receptor related disease or condition” includes a disease or condition in a subject, and preferably a human subject for which administration of a serotonin receptor is beneficial, or for which modulation of the serotonin receptor's activity results in improved outcomes in the subject.

The subject can be any animal, domestic, livestock or wild, including, but not limited to cats, dogs, horses, pigs and cattle, and preferably human subjects, and even more preferably a human subject suffering from, or at risk of suffering from one or more serotonin receptor related diseases or conditions. As used herein, the terms patient and subject may be used interchangeably.

“Pharmaceutical compositions” are compositions that include an amount (for example, a unit dosage) of one or more of the disclosed compounds together with one or more non-toxic pharmaceutically acceptable carriers, including additives, diluents, and/or adjuvants, and optionally other biologically active ingredients. Such pharmaceutical compositions can be prepared by standard pharmaceutical The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, diluents, excipients, etc. can be found in standard pharmaceutical texts. See, for example, “Handbook of Pharmaceutical Additives”, 2nd Edition (eds. M. Ash and I. Ash), 2001 (Synapse Information Resources, Inc., Endicott, N.Y., USA), “Remington's Pharmaceutical Sciences”, 20th edition, pub. Lippincott, Williams & Wilkins, 2000; and “Handbook of Pharmaceutical Excipients”, 2nd edition, 1994.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary, shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

The present invention proposes the use of a combination of drug dosage in a two part pill or capsule where the time release properties of the two compounds are controlled. To compensate for the different timing of the compounds to reach full physiological effect, these properties can be held static or delivered with an enhanced bioavailability formulation. Additionally, the present invention proposes that the delivery of the entactogen component may be such that the anxiolytic effect may be present prior to and sustained until post intoxication with the psilocybin or tryptamine component.

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrants will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet. Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet. Other conventional ingredients include anti-oxidants, colorants, flavoring agents, preservatives and taste-masking agents. Exemplary tablets contain up to about 80 wt % drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tableting. The final formulation may include one or more layers and may be coated or uncoated; or encapsulated. The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0-8247-6918-X), the disclosure of which is incorporated herein by reference in its entirety. Solid formulations for oral administration may be formulated to be immediate and/or modified release. Modified release formulations include delayed-, sustained-, pulsed-, controlled, targeted and programmed release. Suitable modified release formulations are described in U.S. Pat. No. 6,106,864. Details of other suitable release technologies such as high energy dispersions and osmotic and coated particles can be found in Verma et al, Pharmaceutical Technology On-line, 25(2), 1-14 (2001). The use of chewing gum to achieve controlled release is described in WO 00/35298. The disclosures of these references are incorporated herein by reference in their entireties.

Formulations suitable for topical administration (e.g., transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include lozenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active compound. Formulations suitable for administration by inhalation include those presented as an aerosol spray from a pressurized pack, with the use of a suitable propellant, such as dichlorodifluoromethane, trichlorofluoromethane, dichorotetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Suitable emulgents and emulsion stabilizers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams, or spray formulations containing in addition to the active compound, such carriers as are known in the art to be appropriate.

Formulations suitable for parenteral administration (e.g., by injection, including cutaneous, subcutaneous, intramuscular, intravenous, transdermal, and intradermal), include aqueous and non-aqueous isotonic, pyrogen-free, sterile injection solutions which may contain anti-oxidants, buffers, preservatives, stabilizers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

The term “modulation” or “modulate” as used herein in refers to a change in the state away from its current state in response to a composition of the invention, or the state that would occur in the absence of a composition of the invention. For example, the context of a serotonin, or other receptor binding, refers to a change in activation state as compared to the absence of a compound of the invention, or a patent compound of one or more of the compounds of the invention. In one embodiment, modulation of a serotonin, such as 5-HT2A may by effectuated by an agonist compounds and result in its activation. For example, in the context of a inhibiting metabolism, it means a reduction in the glucuronidation of psilocin in a subject. For example, in the context of a inhibiting a physiological reaction to a psychedelic compositions, it means a reduction in the activity of monoamine oxidase and concurrent increase in serotonin level in a subject in response to a composition of the invention.

As used herein, “inhibits,” “inhibition” refers to the decrease relative to the normal wild-type level, or control level. Inhibition may result in a decrease in the metabolic breakdown in a subject, in response a composition of the invention by less than 10%, 10%, 15%, 20%, 25%, 30%, 35%, 40%, 45%, 50%, 55%, 60%, 65%, 70%, 75%, 80%, 85%, 90%, 95% or 100%. In another example, inhibition may result in a decrease, for example inhibition of one or more UGTs that causes the glucuronidation of psilocin in a subject, in response a composition of the invention by less than 10%, 10%, 15%, 20%, 25%, 30%, 35%, 40%, 45%, 50%, 55%, 60%, 65%, 70%, 75%, 80%, 85%, 90%, 95% or 100%. In another example, inhibition may result in a decrease, for example monoamine oxidase, resulting in the reduction of serotonin metabolism or degradation in a subject in response a composition of the invention by less than 10%, 10%, 15%, 20%, 25%, 30%, 35%, 40%, 45%, 50%, 55%, 60%, 65%, 70%, 75%, 80%, 85%, 90%, 95% or 100%. In another example, inhibition may result in a decrease, for example monoamine oxidase, resulting in the increase of serotonin levels in a subject in response a composition of the invention by less than 10%, 10%, 15%, 20%, 25%, 30%, 35%, 40%, 45%, 50%, 55%, 60%, 65%, 70%, 75%, 80%, 85%, 90%, 95% or 100%.

The term “pharmaceutically acceptable salt” means a salt which is acceptable for administration to a patient, such as a mammal, such as human (salts with counterions having acceptable mammalian safety for a given dosage regime). Such salts can be derived from pharmaceutically acceptable inorganic or organic bases and from pharmaceutically acceptable inorganic or organic acids. Pharmaceutically acceptable salts also refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt. For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned above are meant to comprise the therapeutically active non-toxic acid and base addition salt forms which the compounds can form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic, and like acids. Conversely, these salt forms can be converted into the free base form by treatment with an appropriate base. The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g., the lithium, sodium, potassium, magnesium, calcium salts and the like, salts with organic bases, e.g., the benzathine, N-methyl-D-glucamine, hydrabamine salts, and salts with amino acids such as, for example, arginine, lysine, and the like.

Relative amounts of the active ingredient, the pharmaceutically acceptable carriers or excipients, and/or any additional ingredients in a pharmaceutical composition of the invention will vary, depending upon the identity, size, and/or condition of the subject treated and further depending upon the route by which the composition is to be administered. The composition may comprise between 0.1% and 100% (w/w) active ingredient.

As noted above, pharmaceutically acceptable carriers or excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, Poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent. Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include ethanol, polyethylene glycol, phenol, phenolic compounds, bisphenol, chlorobutanol, hydroxybenzoate, and phenylethyl alcohol. Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, German® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs which, preferably contain a unit dosage of one or more therapeutic muropeptides. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Solid dosage forms for oral administration include capsules, tablets, pills, powders, and granules. In such solid dosage forms, the active ingredient is mixed with at least one inert, pharmaceutically acceptable carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically, contemplated routes of administration of the compounds and compositions disclosed herein are inhalation and intranasal administration, subcutaneous administration, mucosal administration, transdermal, and interdermal administration. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The terms “approximately” and “about” refer to a quantity, level, value, or amount that varies by as much as 30%, or in another embodiment by as much as 20%, and in a third embodiment by as much as 10% to a reference quantity, level, value, or amount. As used herein, the singular form “a,” “an,” and “the” include plural references unless the context clearly dictates otherwise. In the context of “approximate” receptor activation, this terms means that the receptor activity will generate approximately the same physiological result as compared to a different receptor activation condition.

As used herein the singular forms “a”, “an”, and “the” include plural referents unless the context clearly dictates otherwise. Thus, for example, reference to “a cell” includes one or more cells and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or ±a range of up to 20%, up to 15%, up to 10%, up to 5%, or up to 4%, 3%, 2%, or 1% compared to the specifically recited value. In addition, the term “between” includes all ranges within the stated number range provided. For example, throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The invention described herein suitably may be practiced in the absence of any element(s) not specifically disclosed herein. Thus, for example, in each instance herein any of the terms “comprising”, “consisting essentially of”, and “consisting of” may be replaced with either of the other two terms. All ratios provided herein as considered approximate.

1. A pharmaceutical composition comprising:
a therapeutically effective amount of:
a psychedelic compound;
one or more secondary agents that inhibit the metabolism of said psychedelic compound, or that inhibit the physiological effect of said psychedelic compound in a subject; and
a pharmaceutically acceptable carrier.

2. The composition of claim 1, wherein said psychedelic compound is an isolated psychedelic compound, or serotonin receptor agonist.

3. The composition of claim 1, wherein said psychedelic compound is a serotonin receptor agonist.

4. The composition of claim 3, wherein said serotonin receptor agonist is an isolated serotonin receptor agonist, or a 5-HT2A serotonin receptor agonist.

5. (canceled)

6. The composition of claim 3, wherein said serotonin receptor agonist is selected from the group consisting of: psilocybin, psilocin, a combination of psilocybin and psilocin, or an pharmaceutically acceptable salt thereof.

7-8. (canceled)

9. The composition of claim 6, wherein said psilocybin or psilocin comprises synthetic psilocybin, synthetic psilocin, or a combination of the same.

10. The composition of claim 6, wherein said psilocybin or psilocin comprises isolated psilocybin, isolated psilocin, or a combination of the same.

11. The composition of claim 1, wherein said secondary agent comprises a uridine 5′-diphospho-glucuronosyltransferase (UGT) inhibitor.

12. The composition of claims 1, wherein said UGT inhibitor inhibits glucuronidation of psilocin in said subject.

13. The composition of claim 11, wherein said UGT inhibitor is selected from the group consisting of: a UG1A10 inhibitor, UGT1A9 inhibitor, or a combination of the same.

14. The composition of claim 11, wherein said UGT inhibitor is selected from the group consisting of: dapagliflozin, canagliflozin, probenecid, sulfinpyrazone, lamotrigine, atazanavir, gemfibrozil, indinavir, valproic acid, p-(di-n-propyl sulphamyl)-benzoic acid (probenecid), 5,7-dihydroxyflavone (chrysin), 5-(2,4-difluorophenyl)-2-hydroxybenzoic acid (diflunisal), 2-((2,3-dimethylphenyl)amino)benzoic acid (mefenamic acid), (2R,3R)-3,5,7-trihydroxy-2-[(2R,3R)-3-(4-hydroxy-3-methoxyphenyl)-2- hydroxymethyl)-2,3-dihydrobenzo[b][1,4]dioxin-6-yl]chroman-4-one (silibinin), 5,6,7,8-tetramethoxy-2-(4-methoxyphenyl)-4H-1-benzopyran-4-one 5,6,7,8,4-pentamethoxyflavone (tangeretin), 1-acetyl-4-(4{[2R,4S)-2-(2,4-dichlorophenyl)-2-(1H-imidazol-1-ylmethyl)-1,3-dioxolan-4-yl]methoxy} phenyl)piperazine (ketoconazole), 1-(butan-2-yl)-4-{4-[4-(4-{[(2R,4 S)-2-(2,4-dichlorophenyl)-2-(1H-1,2,4-triazol-1-ylmethyl)-1,3 -dioxolan-4-yl]methoxy}phenyl)piperazin-1-yl]phenyl}-4,5-dihydro-1H-1,2,4-triazol-5-one (itraconazole), 5-thiazolylmethyl ((alphaS)-alpha-((1S,3S-1-hydroxy-3-((2S)-2-(3-((2-isopropyl-4-thiazolyl)methyl)-3-methylureido)-3-methylbutyramido)-4- phenylbutyl)phenethyl)carbamate (ritonavir), 5-((3,4-dimethoxyphenethyl)methylamino)-2-(3,4-dimethoxyphenyl)-2-isopropylvaleronitrile (verapamil), (+)-dipentene (D-limonene), 2′,4′,5′,7′-tetrabromo-4,5,6,7-tetrachlorofluorescein (cyanosine), bilirubin, (5α,14β,18R)-17-(cyclopropylmethyl)-18-[(1S)-1-hydroxy-1,2,2-trimethylpropyl]-6-methoxy-18,19-dihydro-4,5-epoxy-6,14-ethenomorphinan-3-ol (buprenorphine), (22R,25R)-3β-hydroxy-5α-spirostan-12-one (hecogenin), 1-napthol, 2-{[3-(trifluoromethyl)phenyl]amino}pyridine-3-carboxylic acid (niflumic acid), and 2-(2-((2,6-dichlorophenyl)amino)phenyl)acetic acid (diclofenac).

15. The composition of claim 1, wherein said secondary agent comprises an monoamine oxidase (MAO) inhibitor (MAOI).

16. The composition of claim 15, wherein said MAOI comprises an MAO-A inhibitor, or a MAO-B inhibitor, or a combination of the same.

17. The composition of claim 15, wherein said MAOI is selected from the group consisting of: b-carbolines class of inhibitors, tryptoline, pinoline, selegiline, phenelzine, tranylcypromine, hydroxymethyl-beta-carboline, oclobemide, harmane, harmine, luteolin, quercetin, flavonols and flavones and flavonoids, amiflamine, brofaromine, clorgyline, alpha-ethyltryptamine, iproclozide, iproniazid, isocarboxazid, mebanazine, moclobemide, nialamide, pargyline, pheniprazine, pirlindole, safrazine, toloxatone, and tranlcypromine.

18. (canceled)

19. The composition of claim 1, wherein said therapeutically effective amount comprises a non-intoxicating, or sub-intoxicating dose.

20. The composition of claim 19, wherein said non-intoxicating, or sub-intoxicating dose comprises a nanomolar dose of said psychedelic compound.

21. The composition of claim 1, wherein said therapeutically effective amount comprises a dose of said psychedelic compound that generates an approximately similar physiological response in a subject that is less than the dose required in the absence of said one or more secondary agents.

22. The composition of claim 1, wherein said one or more secondary agents comprise a UGT inhibitor, and a MAOI, or a combination of the same.

23-32. (canceled)

33. A pharmaceutical composition comprising:
a therapeutically effective amount of:
an isolated psychedelic compound selected from the group consisting of psilocybin, psilocin, or a combination of the same;
a UGT inhibitor that inhibits glucuronidation said psilocin in said subject;
a MAOI; and
— a pharmaceutically acceptable carrier.

34. A pharmaceutical composition comprising:
a therapeutically effective amount of:
an isolated psychedelic compound selected from the group consisting of psilocybin, psilocin, or a combination of the same;
a UGT inhibitor that inhibits glucuronidation said psilocin in said subject; and
a pharmaceutically acceptable carrier.

35-72. (canceled)
